(19) United States
(12) Patent Application Publication
(10) Pub. No.: US 2005/0182315 A1
(43) Pub. Date: Aug. 18, 2005

(54) Magnetic resonance imaging and magnetic navigation systems and methods

(76) Inventors: Rogers C. Ritter, Charlottesville, VA (US); Raju R. Viswanathan, St. Louis, MO (US); Roger N. Hastings, Plymouth, MN (US)

Correspondence Address: HARNESS, DICKEY, & PIERCE, P.L.C, 7700 BONHOMME, STE 400, ST. LOUIS, MO, 63105, US

(21) Appl. No.: 10/983,963

(22) Filed: Nov. 8, 2004

Related U.S. Application Data
(60) Provisional application No. 60/518,496, filed on Nov. 7, 2003.

(51) Int. Cl. A61B 5/055

(52) U.S. Cl. 600/411

(57) ABSTRACT

A system for magnetically imaging an operating region in a subject and magnetically navigating a medical device within the operating region includes a first magnet for applying a static magnetic field to the operating region of sufficient strength for magnetically imaging the operating region and sufficiently strong to permit a medical device to be oriented in the operating region by creating a magnetic moment at the distal end of the medical device, and a second magnet for applying a static magnetic field to the operating region of sufficient strength for magnetically imaging the operating region and sufficiently strong to permit a medical device to be oriented in the operating region by creating a magnetic moment at the distal end of the medical device. In an alternate construction, the system includes a first magnet that is movable between a first position to apply a first static magnetic field to the operating region and a second position to apply a second static magnetic field to the operating region. The method of the invention includes applying a first static magnetic field to the operating region and MR imaging and magnetically navigating a device in the first static field, and then applying a second static magnetic field to the operating region, in a different direction than the first direction, and MR imaging and magnetically navigating a device in the second static field.

### CROSS-REFERENCE TO A PRIOR APPLICATION

This application claims priority of U.S. Patent Application Ser. No. 60/518,496, filed Nov. 7, 2003, the disclosure of which is incorporated by reference.

### BACKGROUND OF THE INVENTION

This invention relates to the MR imaging of, and magnetic navigation of devices in, an operating region in a subject.

MR imaging provides high quality medically useful images of regions of the body without exposure of the subject or the physician to x-rays. Magnetic navigation of devices allows fast and accurate control of very small, highly flexible devices by selectively changing an externally applied magnetic field. It has been proposed to use the static magnetic field of an MR imaging system to magnetically navigate devices in an operating region in a subject by selectively changing a magnetic moment at the distal end of the device, typically by changing the currents in coils disposed in the end of the device. Examples of such devices are disclosed in Kuhn, U.S. Pat. No. 6,216,026, Arenson, U.S. Pat. No. 6,304,769, and Hastings et al., U.S. Pat. No. 6,401,723, the disclosures of all of which are incorporated herein by reference.

A somewhat surprising limitation on the magnetic navigation of medical devices using a static magnetic field is the existence of a “forbidden” plane in which the medical device cannot be directly magnetically turned. This “forbidden” plane is perpendicular to the direction of the static magnetic field, and it is not possible to magnetically orient a medical device in a direction with a component in this plane. Other apparatus or techniques are required to provide for navigation in a static magnetic field, such as from an MR imaging system.

### SUMMARY OF THE INVENTION

The present invention relates to systems for, and methods of, the MR imaging of, and magnetic navigation in, an operating region in a patient using the same static magnetic field.

According to a first embodiment of this invention, at least two magnets are provided for generating static magnetic fields in at least two different directions. Each field is sufficiently strong and uniform over the operating region for MR imaging and for magnetic navigation. At least a first magnet is used to create a first static field sufficient for MR imaging of the operating region, and for navigating in the operating region. When it is desired to magnetically navigate in a direction with a component in a plane perpendicular to the first static field, at least a second magnet is used to create a second static field sufficient for MR imaging of the operating region, and for navigating in the operating region so that MR imaging and magnetic navigation can continue (because the forbidden plane of the second static field is different from the forbidden plane of the first static field).

According to a second embodiment of this invention at least a first magnet is used to create a first static field sufficient for MR imaging of the operating region, and for navigating in the operating region. When it is desired to magnetically navigate in a direction with a component in a plane perpendicular to the first static field, the magnet(s) are moved to create a second static field sufficient for M imaging of the operating region, and for navigating in the operating region so that MR imaging and magnetic navigation can continue (because the forbidden plane of the second static field is different from the forbidden plane of the first static field).

According to a third embodiment of this invention, at least a first magnet is used to create a first static field sufficient for MR imaging of the operating region, and for navigating in the operating region. When it is desired to magnetically navigate in a direction with a component in a plane perpendicular to the applied static field, the subject support is moved to change the direction of the magnetic field with respect to the operating region, and thus the orientation of the forbidden plane with respect to the operating region.

Thus a combined MR imaging and magnetic navigation system can be provided that is not limited to navigations that don't involve orientation in the “forbidden” plane. These and other features and advantages will be in part apparent and in part pointed out hereinafter.

### BRIEF DESCRIPTION OF THE DRAWINGS

FIG. 1 is a schematic view of a combined MR imaging and magnetic navigation system in accordance with a first preferred embodiment of this invention;

FIG. 2 is a schematic view of an alternative construction of the MR imaging and magnetic navigation system in accordance with a first preferred embodiment of this invention;

FIG. 3 is a schematic view of an alternative construction of the MR imaging and magnetic navigation system in accordance with a first preferred embodiment of this invention;

FIG. 4 is a schematic view of an alternative construction of the MR imaging and magnetic navigation system in accordance with a first preferred embodiment of this invention;

FIG. 5 is a schematic view of a combined MR imaging and magnetic navigation system in accordance with a second preferred embodiment of this invention;

FIG. 6 is a schematic view of a combined MR imaging and magnetic system in accordance with a third embodiment of this invention;

FIG. 7 is a schematic view of a first alternate construction of a combined MR imaging and magnetic navigation system in accordance with a third embodiment of this invention; and

FIG. 8 is a schematic view of a second alternate construction of a combined MR imaging and magnetic navigation system in accordance with a third embodiment of this invention.

### DETAILED DESCRIPTION OF THE INVENTION

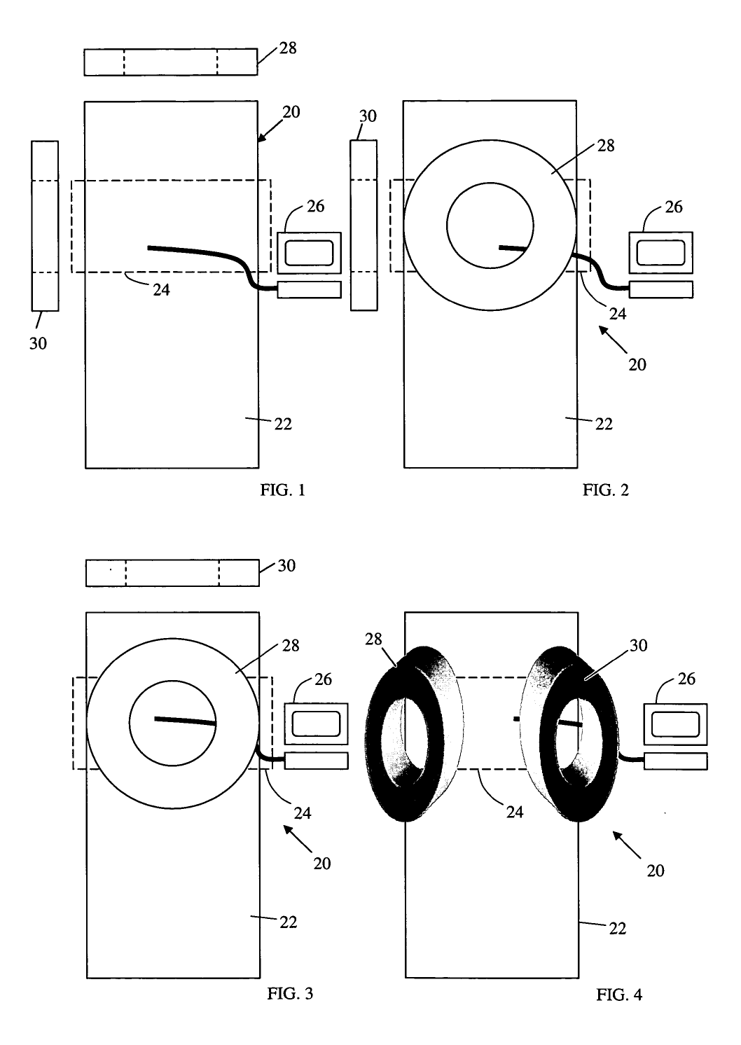

A combined MR imaging and magnetic navigation system in accordance with a first preferred embodiment of this invention is shown schematically as 20 in FIG. 1. As shown in FIG. 1, the system 20 comprises a subject support 22 for supporting a subject during the imaging and navigation procedures. The system 20 also comprises an MR imaging apparatus 24 for imaging the operating region with a static field applied to the operating region. The system 20 also comprises magnetic navigation system 26 for selectively controlling the magnetic moment generated at the distal end of the medical device to orient the distal end of the medical device with respect to the static magnetic field. The system also 20 comprises at least at least first and second magnets for generating static magnetic fields in at least two different directions. Each field is sufficiently strong and uniform over the operating region for MR imaging and for magnetic navigation. At least a first magnet 28 is used to create a first static field sufficient for MR imaging of the operating region in a subject on the support 22, and for navigating in the operating region. In this preferred embodiment the first magnet 28 is an electromagnet, and preferably a superconducting electromagnetic coil. The use of electromagnets allows the magnet to be turned off by turning off the current to the coil. Of course additional magnets can be used to generate the first field, the additional magnets can include stationery and movable permanent magnets and electromagnets. When it is desired to magnetically navigate in a direction with a component in a plane perpendicular to the first static field, at least a second magnet 30 is used to create a second static field sufficient for MR imaging of the operating region, and for navigating in the operating region so that MR imaging and magnetic navigation can continue (because the forbidden plane of the second static field is different from the forbidden plane of the first static field). In this preferred embodiment the second magnet 30 is an electromagnet, and preferably a superconducting electromagnet. The use of electromagnets allows the magnet to be turned off by turning off the current to the coil. Of course additional magnets can be used to generate the first field, the additional magnets can include stationery and movable permanent magnets and electromagnets.

As illustrated in the Figures the first and second magnets 28 and 30 can be arranged in a number of different positions, provided only that theirs fields are not parallel or substantially parallel, so that their respective forbidden planes are different. The positions of the magnets 28 and 30 are preferably chosen so that the forbidden plane of each relative to the anatomy of the subject is such that it is not necessary to frequent shift between the first and second magnets 28 and 30. This speeds up the navigation process, and avoids gaps in the MR imaging. Thus, as shown in FIG. 1, the first magnet 28 is positioned at the end of the patient support 22, and the second magnet 30 is positioned at the side of the patient support. The first magnet 28 generates a first static field B1, with a “forbidden” plane parallel to the transverse or axial plane, and the second magnet 30 generates a field B2, with a “forbidden” plane parallel to the saggital plane. As shown in FIG. 2, the first magnet 28 is positioned above the patient support 22, and the second magnet 30 is positioned at the side of the patient support. The first magnet 28 generates a first static field B1, with a “forbidden” plane parallel to the coronal or frontal plane, and the second magnet 30 generates a second static field B2, with a “forbidden” plane parallel to the saggital plane. As shown in FIG. 3, the first magnet 28 is positioned above the patient support 22, and the second magnet 30 is positioned at the end of the patient support. The first magnet 28 generates a first static field B1, with a “forbidden” plane parallel to the saggital plane, and the second magnet 30 generates a second static field B2, with a “forbidden” plane parallel to the transverse or axial plane. As shown in FIG. 4 the first magnet 28 is mounted at a 45° angle on the left side of the patient support, and the second magnet 30 is mounted at a 45° angle on the right side of the patient support. The first magnet 28 generates a first static magnetic field B1, with a “forbidden” plane parallel to the right anterior oblique plane of the patient, and the second magnet 30 generates a second static magnetic field B2, with a “forbidden” plane parallel to the left anterior oblique plane of the patient.

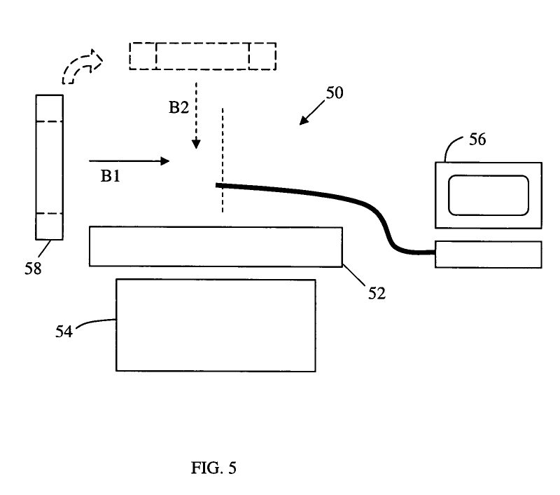

As illustrated in FIG. 5, a second embodiment of this invention indicated generally as 50 comprises a patient support 52. The system 50 also comprises an MR imaging apparatus 54 for imaging the operating region with static field applied to the operating region. The system 20 also comprises magnetic navigation system 56 for selectively controlling the magnetic moment generated at the distal end of the medical device to orient the distal end of the medical device with respect to the static field. The system 50 further comprises at least a first movable magnet 58, movable between at least a first position and a second position. When the magnet 58 is in its first position, it creates a first static field B1 sufficient for MR imaging of the operating region, and for navigating in the operating region. When it is desired to magnetically navigate in a direction with a component in a plane perpendicular to the first static field, the magnet(s) 58 are moved to its second position to create a second static field B2 sufficient for MR imaging of the operating region, and for navigating in the operating region so that MR imaging and magnetic navigation can continue (because the forbidden plane of the second static field is different from the forbidden plane of the first static field). As shown in FIG. 5, the in the first position the magnet 58 creates a static magnetic field B1, with a “forbidden” plane P parallel to the sagittal plane, and in the second position (shown in phantom), the magnet 58 creates a static magnetic field B2, with a “forbidden” plane parallel to the coronal or frontal plane. In this preferred embodiment the first magnet is an electromagnet, and preferably a superconducting electromagnetic coil. The use of electromagnets allows the magnet to be turned off by turning off the current to the coil. Of course additional magnets can be used to generate the first field, the additional magnets can include stationery and movable permanent magnets and electromagnets. As shown in the first position,

The magnet(s) 58 are preferably movable so that there is no overlap in the forbidden planes of the two magnetic fields. Alternatively the motions are selected so that the overlap (which would typically be a line), is in a direction with minimal interference with likely navigations.

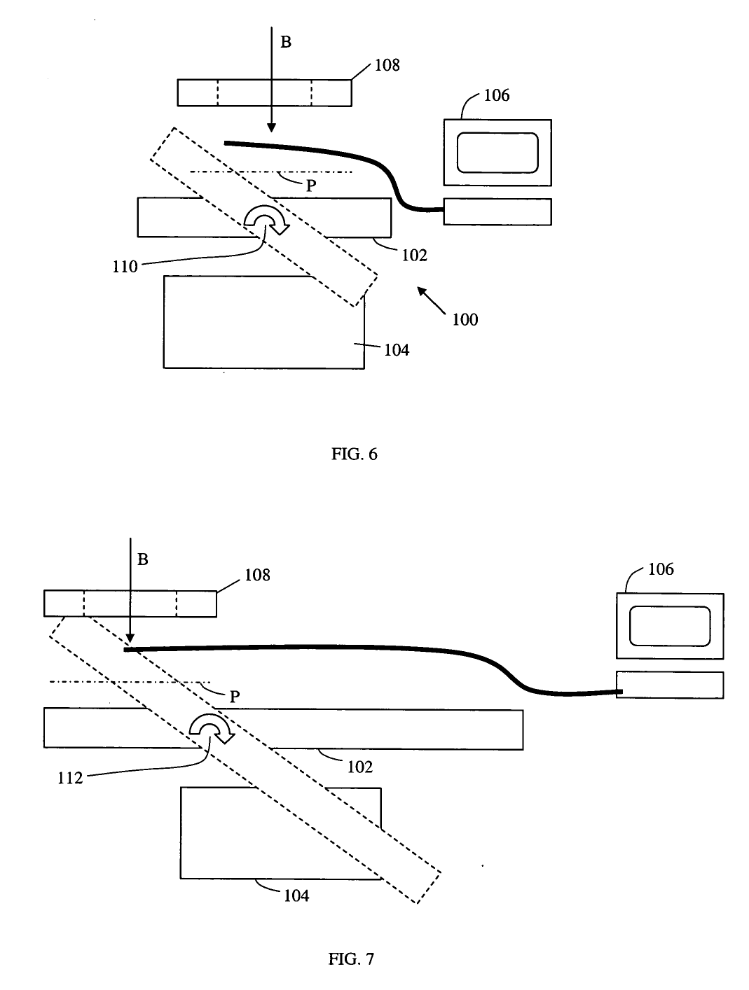

A combined MR imaging and magnetic navigation system in accordance with a third embodiment of this invention indicated generally as 100, is shown in FIGS. 6, 7, and 8. The system 100 comprises a support 102 for supporting a subject during imaging and navigation procedures. The system 100 further comprises an MR imaging system 104 for imaging an operating region with a static magnetic field applied to the operating region. The system further comprises a magnetic navigation system 106 for selectively controlling the magnetic moment generated at the distal end of the medical device to orient the distal end of the medical device with respect to a static magnetic field applied to the operating region. (This is typically achieved by controlling electric currents in one or more coils in the distal end of the medical device, or otherwise activating and deactivating magnetic elements in the distal end of the medical device.)

The system 100 also includes at least one magnet 106 for applying a static magnetic field in direction B to the operating region. The magnet 106 may be one stationary magnet; it may be multiple magnets, as shown and described in the first embodiment, or there may be one or more moveable magnets, as shown or described in the third embodiment.

In accordance with the principles of this third embodiment, the subject support 102 is pivotable with respect to the magnets 108 to move the subject and thereby change the orientation of the “forbidden” plane P with respect to the subject. The movement is preferably a pivoting motion about an axis extending through or near to the operating region so that while the direction of the static magnetic field B and thus the orientation of the “forbidden” plane P relative to the operating region changes, the field strength remains relatively constant.

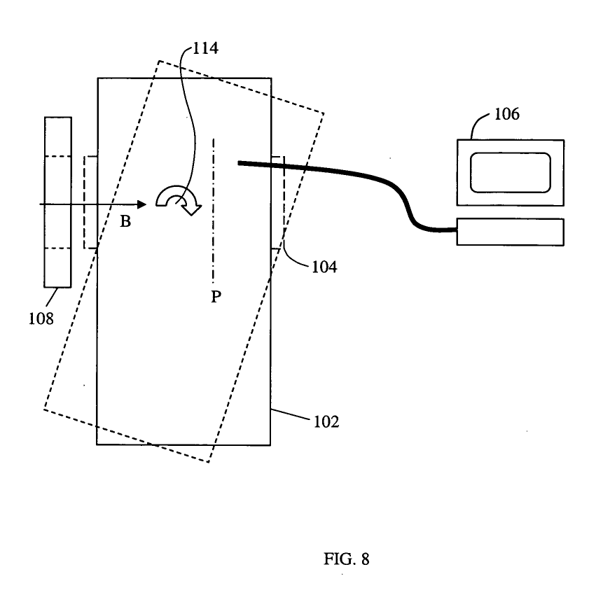

Thus as shown in FIG. 6, the support 102 pivots about an axis 110, parallel to the longitudinal (cranial-caudal) direction. As shown in FIG. 7, the support 102 pivots about an axis 112, parallel to the transverse direction. As shown in FIG. 8, the support 102 pivots about an axis 112, parallel to the anterior-posterior direction. The support 102 preferably implement at least one, but can implement two or all three movements.

Operation

In the first embodiment the first magnet 24 is operating to apply a first static magnetic field to the operating region in a subject on the support 22. This first static field is used for MR imaging and for magnetic navigation. An elongate medical device is oriented with respect to the first static magnetic field by changing the magnetic moment at the tip of the device. Typically the devices is provided with one or more (magnetic) coils (or other magnetic elements) at its distal tip, and the magnetic moment at the tip is controlled by controlling the currents in the coils.

When it is desired to turn the distal end of the device in a direction with a component in the plane perpendicular to the first static magnetic field, the first magnet 24 is turned off, and the second magnet 26 is turned on to establish a second magnetic field in a second direction different from the first direction. The MR imaging can continue, using this second magnetic field, and because the direction of the second field is different from the direction of the first field, the directions of their “forbidden” planes are different, so that navigation can continue as well. Once the turn is completed, the magnets 24 and 26 can be operated to again apply the first static field to the operating region, or the MR imaging and navigation can continue using the second static field until is it is desired to turn the distal end of the device in a direction with a component in the plane perpendicular to the second static magnetic field. Then the second magnet 26 is either turned off, and the first magnet 24 is either turned on to reestablish establish the first magnet field. At least one of the magnets used in to create one of the fields is preferably not used to create the other of the fields. However, it is possible that one of more of the magnets used to create one field are also used to create the other field.

Thus a system for, and a method of MR imaging and magnetic navigation is provided, in which magnetic navigation is not restricted in any particular plane or direction.

In the second embodiment, the magnet 58 is operated to apply a first static field B1 to the operating region in a subject on the support 52. This first static field is used for MR imaging and for magnetic navigation. An elongate medical device is oriented with respect to the first static magnetic field by changing the magnetic moment at the tip of the device. Typically, the device is provided with one or more magnetic coils (or other magnetic elements) at its distal tip, and the magnetic moment at the tip is controlled by controlling the currents in the coils.

When it is desired to turn the distal end of the device in a direction with a component in the plane perpendicular to the first static magnetic field, the magnet 58 is moved to a position to establish a second static magnetic field in a second direction different from the first direction. If the magnet 58 is a permanent magnet the magnet may be moved away from the operating region before it is move, to minimize the effect of the changing field in the operating region. If the magnet 58 is an electromagnet, the magnet may be turned down or turned off before the magnet is moved, to minimize the effect of the changing field in the operating region.

In the third embodiment the magnet 108 is operated to apply a static magnetic field to the operating region in a subject on the support 102. This static field is used for MR imaging and for magnetic navigation. An elongate medical device is oriented with respect to this static field by changing the magnetic moment at the tip of the device. Typically the device is provided with one or more magnetic coils (or other magnetic elements) at its distal tip, and the magnetic moment at the tip is controlled by controlling the currents in the coils.

When it is desired to turn the distal end of the device in a direction with a component in the plane perpendicular to the static magnetic field, the support 102 is moved to change the direction of the static field relative to the operating region, and thereby change the orientation of the “forbidden” plane. If the magnet 108 is a permanent magnet, the magnet may be moved away from the operating region before the support 102 is moved, to minimize the effect of the changing field in the operating region. If the magnet 108 is an electromagnet, the magnet may be turned down or turned off before the magnet is moved, to minimize the effect of the changing field in the operating region.

The motion of the support 102 is preferably about an axis through the operating region so that while the orientation of the “forbidden” plan changes, the distance between the magnet 108 and the operating region does not. Some various in the distance between the magnet 108 and the operating region can be tolerated, and some can be accommodated by movement of the magnet.

Depending upon the motion of the patient, the patient is preferably moved back to the patient's original positions as soon as practical after the desired turn in the “forbidden” plan to avoid discomfort.

1. A method of navigating the distal end of an elongate medical device in an operating region in a subject using a static magnetic field, the method comprising:
applying a static magnetic field in a first direction to the operating region;
magnetically imaging the operating region using the static magnetic field in the first direction;
magnetically navigating the distal end of a medical device in the operating region by selectively creating a magnetic moment in the distal end of the device to orient the distal end with respect to the static magnetic field in the first direction;
applying a static magnetic field in a second direction to the operating region;
magnetically imaging the operating region using the static magnetic field in the second direction; and
magnetically navigating the distal end of a medical device in the operating region by selectively creating a magnetic moment in the distal end of the device to orient the distal end with respect to the static magnetic field in the second direction.

2. The method according to claim 1 wherein the static magnetic field in the first direction is created by at least one magnet not used in creating the static magnetic field in the second direction, and wherein the static magnetic field in the second direction is created by at least one magnet not used in creating the static magnetic field in the first direction.

3. The method according to claim 1 wherein the static magnetic field in the first direction and the static magnetic field in the second direction are applied by at least one coil that is moved between a first position in which the at least coil contributes to the static field in the first direction, and a second position in which the at least one coil contributes to the static field in the second direction.

4. A system for magnetically imaging an operating region in a subject and magnetically navigating a medical device within the operating region, the system comprising: a first magnet for applying a static magnetic field to the operating region of sufficient strength for magnetically imaging the operating region and sufficiently strong to permit a medical device to be oriented in the operating region by creating a magnetic moment at the distal end of the medical device, and a second magnet for applying a static magnetic field to the operating region of sufficient strength for magnetically imaging the operating region and sufficiently strong to permit a medical device to be oriented in the operating region by creating a magnetic moment at the distal end of the medical device.

5. The system according to claim 4 wherein the first and second magnets are permanent magnets.

6. The system according to claim 4 wherein the first and second magnets are electromagnets.

7. The system according to claim 6 wherein the first and second magnets are superconducting electromagnets.

8. A system for magnetically imaging an operating region in a subject and magnetically navigating a medical device within the operating region, the system comprising: a magnet; a support for movable mounting the magnet for movement between a first position in which the magnet applies a static magnetic field in a first direction to the operating region of sufficient strength for magnetically imaging the operating region and sufficiently strong to permit a medical device to be oriented in the operating region by creating a magnetic moment at the distal end of the medical device, and a second position in which the magnet applies a static magnetic field in a second direction to the operating region of sufficient strength for magnetically imaging the operating region and sufficiently strong to permit a medical device to be oriented in the operating region by creating a magnetic moment at the distal end of the medical device.

9. The system according to claim 8 wherein the magnet is a permanent magnet.

10. The system according to claim 8 wherein magnet is an electromagnet.

11. The system according to claim 10 wherein the magnet is a superconducting electromagnet.

12. A method of navigating the distal end of an elongate medical device in an operating region in a subject using a static magnetic field, the method comprising supporting a subject on a moveable support; applying a static magnetic field in a first direction to the operating region;
magnetically imaging the operating region using the static magnetic field; magnetically navigating the distal end of a medical device in the operating region by selectively creating a magnetic moment in the distal end of the device to orient the distal end with respect to the static magnetic field, moving the support relative to the static magnetic field to change the direction of static field relative to the operating region.

13. The method according to claim 12 wherein the support is moved about an axis extending through the operating region.

14. The method according to claim 13 wherein the axis is perpendicular to the direction of the static magnetic field.

15. A method of navigation the distal end of an elongate medical device in an operating region in a subject using a static magnetic field, the method comprising supporting a subject on a support;
applying a static magnetic field to the operating region in first angular relation;
magnetically imaging the operating region using the static magnetic field in its first angular orientation;
magnetically navigating the distal end of a device in the operating region by selectively creating magnetic moment in the distal end of the device to orient the distal end with respect to the static magnetic field in the first angular orientation;
changing the angular relation between the static field and the operating region from the first angular orientation to a second angular orientation;
magnetically navigating the distal end of the medical device in the operating region by selectively creating a magnetic moment in the distal end of the device to orient the distal end with respect to the static magnetic field in its second orientation.

16. The method according to claim 15 wherein changing the angular relation between the static field and the operating region comprises applying the magnetic field in the second orientation with at least one different magnet than is used to apply a magnetic field in the first orientation.

17. The method according to claim 15 wherein changing the angular relation between the static field and the operating region comprises moving at least one magnet used to apply the magnetic field in the first orientation.

18. The method according to claim 15 wherein changing the angular relation between the static field and the operating region comprises moving the patient relative to at least one magnet applying the static field in the first orientation.
